(19) United States
(12) Patent Application Publication
(10) Pub. No.: US 2006/0167482 A1
(43) Pub. Date: Jul. 27, 2006

(54) Device for transfixing and joining tissue

(76) Inventors: Christopher Paul Swain, London (GB); Charles Alexander Mosse, London (GB); Annette Dora Anna Fritscher-Ravens, London (GB); Timothy Noel Mills, London (GB)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/242,713

(22) Filed: Oct. 3, 2005

(30) Foreign Application Priority Data
Apr. 4, 2003 (GB) 0307826.8
Mar. 31, 2004 (WO) PCT/GB04/01350

(51) Int. Cl. A61B 17/08

(52) U.S. Cl. 606/151

(57) ABSTRACT

A device is provided for use in joining together first and second tissue layers. The device comprises a catheter with an inflatable balloon mounted on the catheter adjacent one and thereof. An inflation channel is provided which is an fluid communication with the interior of the balloon and with a source of inflation fluid for introducing the inflation fluid into the interior of the balloon. The balloon is inflated and engages an outwardly facing surface of the first tissue layer and a tissue-engaging member, for example a cup or another balloon, engages an outwardly facing surface of the second tissue layer. The inwardly facing surfaces of the first and second tissue layers are thereby urged into engagement with on another.

### BACKGROUND OF THE INVENTION

This invention relates to methods and devices for transfixing and joining tissues and, more particularly, to forming anastomoses.

In abdominal and vascular surgery anastomosis, the joining together of hollow structures, is, an important goal. The ability to establish continuity between two hollow structures can relieve blockage due to cancer, inflammation or other pathology, can allow the removal of abnormal tissue or organs, and, by bypassing a blocked segment, can allow the unimpeded movement of food or facilitate the flow of blood or bile through the body.

Anastomoses are most commonly formed at open abdominal surgery (laparotomy). Hand sewn anastomoses, usually in two or even three layers, are widely performed but are time consuming and require large incisions for hand access. Stapled anastomoses became widely performed especially in colonic surgery since they allowed surgeons to remove low rectal tumours. The short rectal remnant could be joined to the colon above the tumour at a site where it was difficult to place stitches by hand, and in consequence allowed patients to recover without needing a permanent colostomy. The advent of laparoscopic surgery staplers allowed anastomoses to be formed through incisions of 1-2 cm or so that were just large enough to allow passage of these instruments inside the abdominal or thoracic cavity.

Some aids to form anastomoses have been developed. J. B. Murphy, an American surgeon working in Chicago in the 1880's, popularized surgical anastomoses by creating a compression button device for anastomosis. The device had two mushroom-shaped buttons, which could be placed in the two organs to be joined. The buttons could be pressed together by an internal spring in the stalk of the mushroom and the organs would be welded together by the consequent ischaemia (lack of blood supply) at the sites where the buttons were pressed together. Eventually the button device would fall through into the gut, leaving an anastomosis or hole and be passed through the body into the toilet. Compression button anastomoses are still used at open colonic surgery. The use of magnets to compress tissue to form an anastomosis has also been described, and a spring compression button method using a biofragmentable ring has been employed, especially in the rectum.

An anastomosis procedure has been described in an article entitled “Anastomosis as Flexible Endoscopy: an experimental study of compression button gastrojejunoscopy”, C P Swain and T N Mills, Gastrointestinal Endoscopy 1991, 37: 625-631, in which, as its title implies, a method is described of forming anastomoses using a flexible endoscope. The method described there involved introducing a flexible endoscope into one of the two structures to be joined (in this case the stomach), and entering the second of the two structures (in this case the small bowel) by foaming an incision in the abdomen of the subject. The present invention is directed, in one aspect thereof, to the formation of anastomoses without the need to make such external incisions, though in other aspects thereof the means described herein which make this possible are applied to anastomoses in the formation of which such incisions are made.

The ability to form anastomoses using flexible endoscopic or percutaneous procedures without opening the abdomen or chest or using laparoscopic methods might offer advantages especially to patients with advanced cancer or in elderly or sick patients, who might withstand conventional surgery poorly: In particular, flexible endoscopy might allow anastomoses to be formed from stomach to small bowel, duodenum to gallbladder, and small bowel to colon.

International patent publication PCTIGB02/02168 describes a number of forms of anastomosis device which can be used, inter alia, via an endoscope, and the present application is directed to yet another form of anastomosis device, which can be both simple and effective, and which requires little in the way of novel hardware.

### BRIEF SUMMARY OF THE INVENTION

According to the present invention there is provided a device for use in joining together first and second tissue layers, the device comprising a catheter, an inflatable balloon mounted on the catheter adjacent one end thereof, an inflation channel in fluid communication with the interior of the balloon and with a source of inflation fluid, for introducing the inflation fluid into the interior of the balloon, the balloon being adapted, when inflated, to engage an outwardly facing surface of the first tissue layer and a tissue-engaging member adapted to engage an outwardly facing surface of the second tissue layer, whereby the inwardly facing surfaces of the first and second tissue layers are urged into engagement with one another.

### BRIEF DESCRIPTION OF THE DRAWINGS

In the accompanying drawings:

FIGS. 1 to 7 show diagrammatically successive stages in the formation of an anastomosis using the device according to the present invention;

FIG. 8 shows an alternative pair of anastomosis elements to those shown in the earlier Figures;

FIGS. 9a to 9d, show embodiments of locking elements for use in the invention; and

FIG. 10 shows another alternative type of balloon to that illustrated in FIGS. 1 to 7.

### DETAILED DESCRIPTION OF THE INVENTION

Referring to FIGS. 1 to 7, an anastomosis is foamed as follows between the stomach wall (marked SW) of a patient and the wall of the patient's small bowel (marked SB). It is to be understood, however, that the procedure would be similar if the anastomosis were formed-in some other location, for example between the stomach and the gallbladder.

A hollow needle 1 is passed through the biopsy channel 2 of an echoendoscope 3, indicated in the drawings simply by a block. The echoendoscope 3 is provided with means (not shown) for forming an ultrasound image of the region in which the anastomosis is to be formed. A cylindrical tag 4 is slidably received within the hollow needle 1, and the leading end of a thread 5 is attached to the tag 4. The thread passes out of the hollow needle, and thence out through the patient's mouth. The drawing shows the forward end of the needle partially cut away, so that the thread 5, but not tag, can emerge through this cutaway portion. However, it is alternatively possible for this cutaway portion to be omitted, in which case the thread emerges from the forward end of the needle and doubles back over the outside of the forward end on its way to the patient's mouth. As shown in FIG. 1, the needle is passed through the stomach wall and its tip passes through the wall of the small bowel.

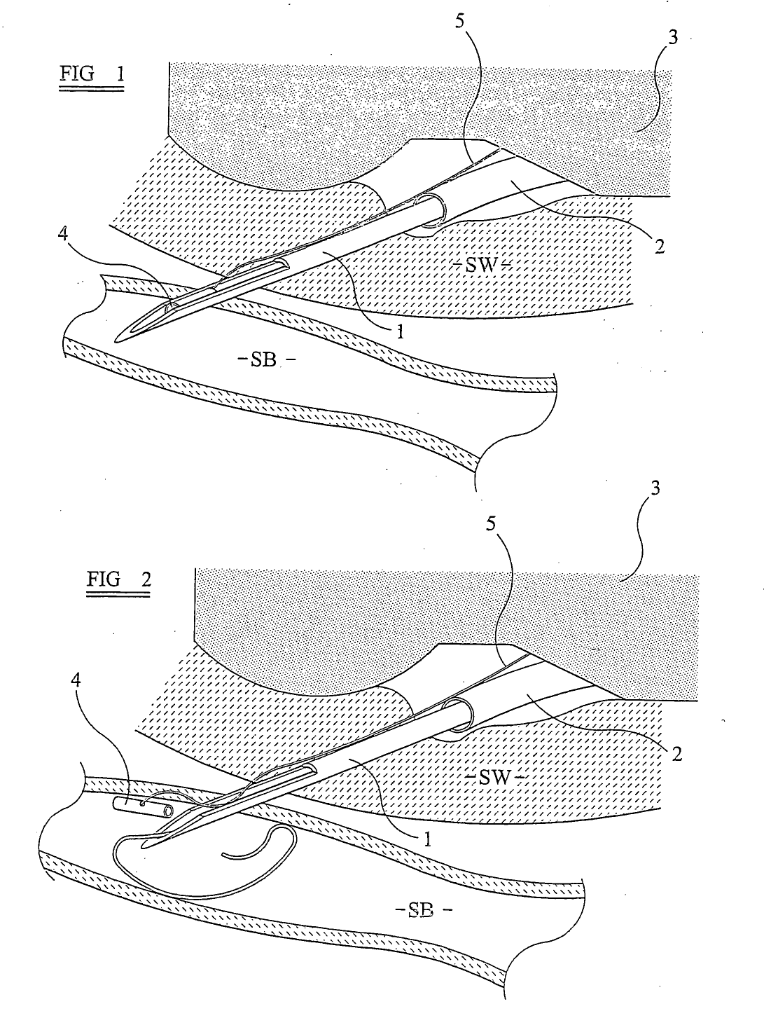

As shown in FIG. 2, the tag 4 is expelled from the end of the hollow needle 1. This is achieved using a pushing rod (not shown) which is passed through the hollow needle behind the tag, and is then withdrawn once the tag has been expelled from the needle. Following expulsion of the tag from the hollow needle, a guide wire 6 is passed through the hollow needle, so that its leading end emerges into the small bowel.

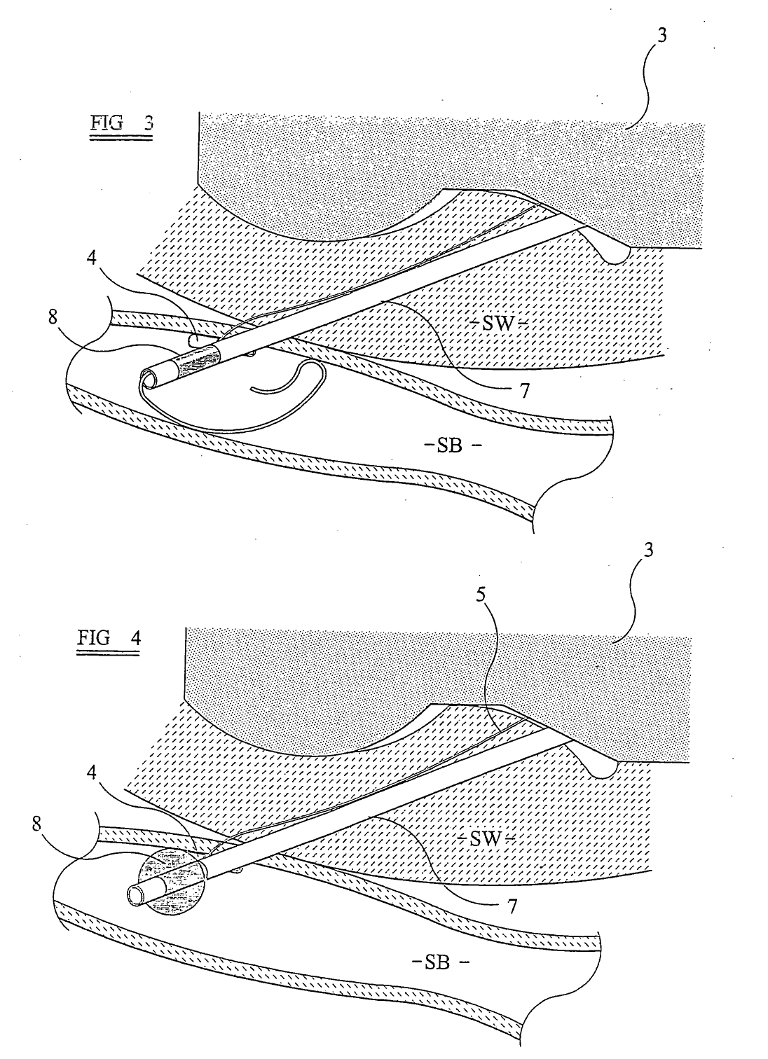

The needle is then withdrawn, leaving the guide wire passing through the wall of the stomach and into the small bowel. Then, as shown in FIG. 3, a catheter 7, with a low profile balloon 8 surrounding a portion of the catheter adjacent its tip, is passed over the guide wire, and down through the biopsy channel 2, so that the balloon-carrying part of the catheter 7 passes through the wall of the stomach and into the small bowel. During passage of the catheter through the stomach wall and into the small bowel, a tension is applied to the thread 5 by the person performing the anastomosis procedure, so that the tag 4 pulls the wall of the small bowel into firm contact with the stomach wall in the region through which the catheter 7 is to pass. Although the tag 4 may not always be essential, it is at least helpful in ensuring easier passage of the catheter to the position shown in FIG. 3.

As is conventional with balloon catheters, an inflation channel runs within it, separate from the channel through which the guide wire passes, and it is through the inflation channel that a fluid is passed into the balloon 8 to cause inflation thereof. FIG. 4 of the drawings shows the balloon in its inflated state.

There are various possibilities are regards the fluid. The fluid could be a gas, for example air, but preferably it is a liquid, since it is easier to avoid leakage with a liquid. Where a liquid is used this could be water, saline, or some other physiologically acceptable liquid. There are, however, other possibilities. In particular, a material can be used to inflate the balloon which is in liquid form at the time it is introduced, but which subsequently becomes solid. For example, an epoxy resin might be introduced into balloon to inflate it, and then left for some minutes to solidify, or it, might be possible to solidify the resin by subsequent introduction of a hardener. Alternatively, the material introduced, which might again be an epoxy resin, could be hardened by the use of heat. Such heat could be applied to the liquid in various ways, including:

(i) Providing the device with an optical fiber which extends from a light source exterior of the patient, through the catheter 7, to the interior of the balloon, where the tip of the optical fiber is blackened so that the line reaching it is converted to heat.

(ii) If the liquid that is to be hardened is electrically conductive, it could be heated by locating in the interior of the balloon a bipolar electrode, the individual electrodes of which are spaced apart by a short distance, e.g. 2 mm, the electrode being connected to an external source of electrical power via electrical conductors which run through the catheter 7.

(iii) As in (ii) but replacing the bipolar electrode with a diode, resistor, thennistor or other dissipative electrical component.

(iv) Running a coaxial cable from an external microwave source through the catheter 7 into the interior of the balloon, the frequency of the microwaves being chosen to be such as to cause heating of the particular liquid within the balloon.

Examples of suitable materials are disclosed in WO 01/50974, to which attention is directed. The light could be introduced into the liquid through an optical fibre arranged as in (i) above, but with the blackened tip omitted.

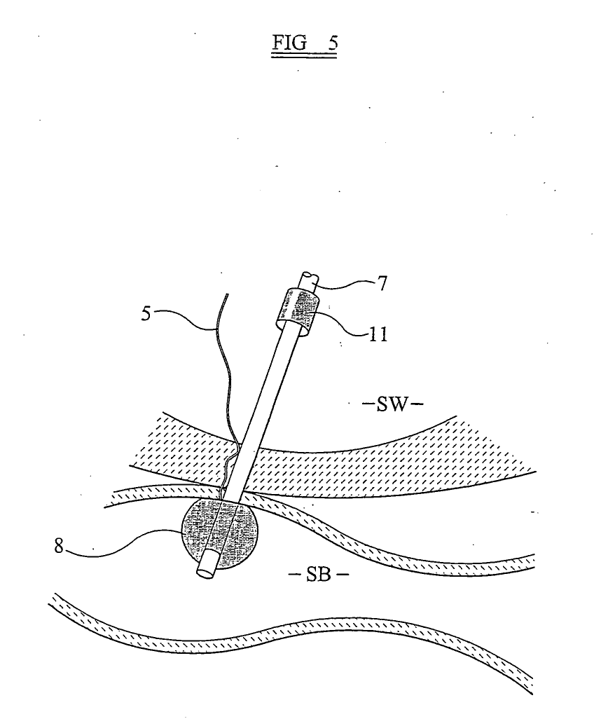

Once the balloon is inflated it is pulled back, by pulling on the balloon catheter, so as to press the stomach wall and the small bowel wall together very firmly. This is as shown in FIG. 5, which also shows an element 11 fixed to the exterior of the catheter. The purpose of the element 11 is referred to in more detail below.

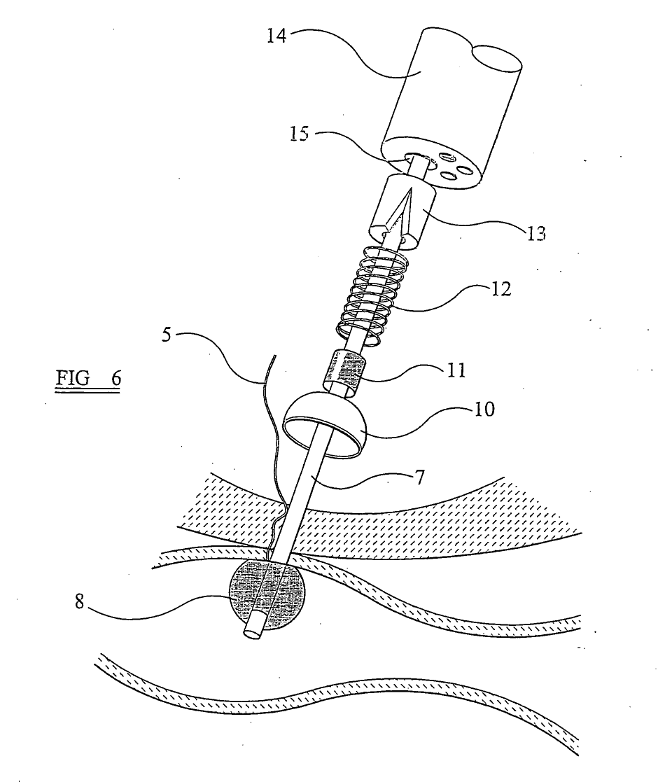

A cup-shaped compression device 10, which can be seen in FIG. 6, is then passed down over the catheter 7 and over the element 11, which is an inner locking element. The compression device is in the form of approximately a hemisphere, with an aperture therein of a size larger than the external diameter of the inner locking element 11. This is followed by a compression spring 12 and an outer locking element 13. The elements 10, 12, and 13 are pushed down the catheter 7 by the leading end of an endoscope 14, along whose biopsy channel 15 the catheter 7 passes. For this purpose, the external diameter of the outer locking element 13 must be larger in diameter than the biopsy channel 15.

The endoscope could be the same as the endoscope 3 referred to above, or it could be different. In the drawings, two different endoscop.es are shown, which have different view systems at their forward ends. Although this has an advantage in that the best view system for the first part of the procedure may not be the best for the latter part of the procedure, it is of course simpler to use the same endoscope throughout. If that is done it may further be advantageous not to have to withdraw the endoscope after the first part of the procedure and reinsert it for the latter part, something which is necessary when practising the embodiment shown in FIGS. 1 to 7 and 9, because the compression device 10 and the locking element 13 are too large to pass through the biopsy channel 15. To avoid this problem the compression device can be made in the form of an umbrella, which can be folded in order to pass it through the biopsy channel and then unfolded for use as a compression device. Further, the locking element 13 can be made smaller, so that it will pass through the biopsy channel, and then the locking element 13 can be pushed into engagement with the locking element 11 (described below), not by the end of the endoscope but by an auxiliary pushing catheter which can be passed down the biopsy charnel over the catheter 7.

By exerting force on the outer locking element 13, by means of the leading end of the endoscope 14, the cup-shaped compression element is caused to compress the tissue which it engages, so that it becomes one element of an anastomosis-forming device, the other element being constituted by the balloon 8. The spring 12 is forced into compression, and the outer locking element 13 is a snap fit on the inner locking element 11. It may sometimes be possible to omit the spring 12 where the balloon is inflated with gas or a liquid which does not solidify and this can function, at least to some extent, as a spring.

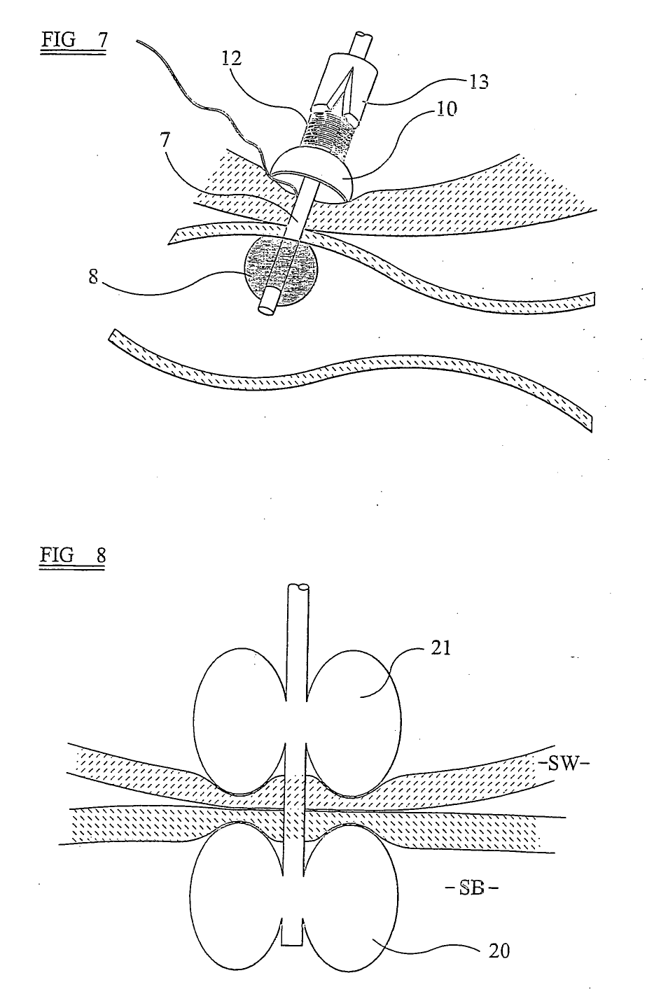

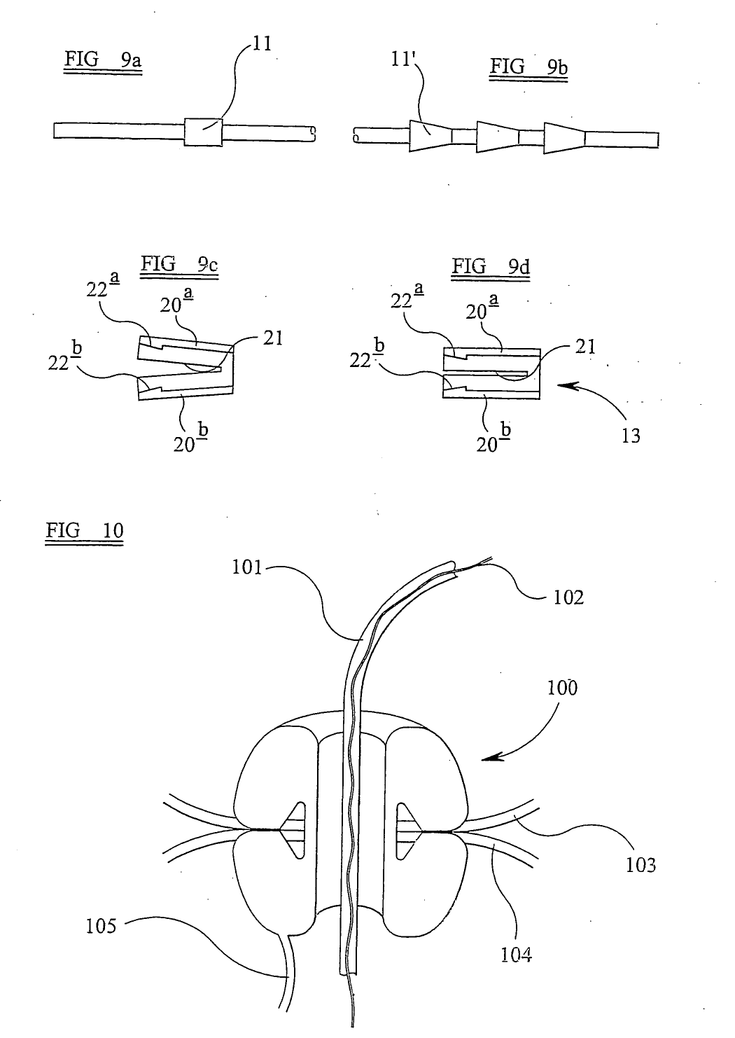

Embodiments of the locking elements 11 and 13 are shown in FIGS. 9a to 9d. FIG. 9b shows the locking element 13 in its rest position, where it can be seen to comprise two legs 20a and 20b separated by a slit and terminating in ratchet teeth 22a and 22b. The locking element 13 is made of a material, for example a plastics material, which is sufficiently resilient to allow the locking element 12 to force the legs apart, as shown in FIG. 9c, to allow the locking element to pass beyond the ratchet teeth. Once the locking element has passed beyond those teeth they spring back and prevent the locking element 11 being withdrawn past them. If desired, a plurality of locking elements 11 can be provided along part of the length of the exterior of the catheter 7, so that the locking element 13 can be held in different positions, depending on the thickness of the tissue layers to be compressed, and the extent to which it is desired to compress those layers. The plurality of locking elements can be integrated into a single component, as indicated in FIG. 9d by reference 11′.

If the liquid in the balloon is one which does not solidify, e.g. water, the catheter is then sealed, so that the balloon cannot deflate. There are various ways of sealing the liquid inside the balloon. One way of providing sealing is for there to be a one-way valve upstream of the balloon. Another possibility is to fill the catheter lumen with a hot melt, which will seal the water in the balloon when it has cooled and solidified. Yet another possibility is to make a heat seal by simultaneously compressing and heating the catheter, and this could both seal liquid in the balloon and cut the catheter.

When the catheter has not been cut in the course of sealing it, it can be subsequently cut with a guillotine or hot wire in a tube. The situation is then as illustrated in FIG. 7. Another possible way of separating the distal part of the catheter 7 from the proximal part is to form a line of weakness in the catheter at the location where separation is required, and then push the two portions apart by pushing on the locking element 13 by means of the distal end of the endoscope or a pushing catheter running through that channel over the catheter 7. Instead of a line of weakness, the catheter 7 can initially be made of two sections joined by a connector, e.g. a sleeve, which holds the two sections together only sufficiently securely to prevent their accidentally separating. The two sections can then be separated in the way just described for the case where a line of weakness is provided.

Although the drawing shows a generally spherical balloon, there are alternative balloon shapes which may be useful. One such shape is a dumbbell, and when a balloon of that shape is used in place of the balloon 8, it maybe advantageous for the second anastomosis element also to be a dumbbell-shaped balloon, rather than being cup-shaped, as illustrated in FIGS. 1 to 7. FIG. 8 shows such a pair of dumbbell balloons in the position which they adopt when they are forming the anastomosis, i.e. when the tissue is compressed between them. As shown in FIG. 8, there is a first pair of dumbbell-shaped balloons 20, replacing balloon 8, and a second pair of dumbbell-shaped balloons 21, replacing the cup-shaped element 10. It will be seen that these pairs of balloons form an annular region of compressed tissue, which is what is required for an anastomosis to be formed. It will be appreciated that, in use of the arrangement shown in FIG. 8, the catheter is initially placed in position and the balloons 20 are then inflated. Once they have been inflated the catheter can be pulled back to cause the inflated balloons 20 to exert pressure on the tissue, and the balloons 21 can then be inflated to press the tissue from the other side.

Yet another possibility is to use a balloon whose shape, when inflated, is as shown in FIG. 10, and has a lumen which is larger than the catheter to which it is attached. The aim of using a balloon of this type is to obtain a larger anastomosis than would otherwise be possible. FIG. 10 shows a balloon 100 whose shape is that of two interconnected toroids. It surrounds a catheter 101 through which is threaded a guide wire 102. The balloon is shown compressing two layers of tissue 103 and 104. The balloon is inflated via an inflation tube 105, separate from the catheter 101. For the purpose of introducing the balloon, it can be folded on the outside of the catheter and adhered to one side thereof. If the degree of adherence is low, it will separate from the catheter on expansion, and the catheter can then be removed, leaving the balloon in the desired position in relation to the tissue layers. Alternatively, a larger catheter could be slid over the folded balloon, the larger catheter being withdrawn once the balloon is in place. In either event, the inflation tube 105 must subsequently be severed, after the balloon has been sealed, or after the liquid in the balloon has become solid, as the case may be.

It is also to be noted that when reference is made herein to the inflatable balloon mounted on the catheter adjacent one end thereof, it is to be understood that the balloon might be right at the tip of the catheter, even to the extent that the leading end of the balloon is a short distance, say 5 mm, beyond the leading end of the catheter. Alternatively, the catheter might have a balloon-free lead-in section extending, say, 20 mm or even more, beyond the leading end of the balloon.

1. A device for use in joining together first and second tissue layers, the device comprising a catheter, an inflatable balloon mounted on the catheter adjacent one end thereof, an inflation channel in fluid communication with the interior of the balloon and with a source of inflation fluid, for introducing the inflation fluid into the interior of the balloon, the balloon being adapted, when inflated, to engage an outwardly facing surface of the first tissue layer and a tissue-engaging member adapted to engage an outwardly facing surface of the second tissue layer, whereby the inwardly facing surfaces of the first and second tissue layers are urged into engagement with one another.

2. A device according to claim 1, wherein the inflation fluid is a gas.

3. A device according to claim 1, wherein the inflation fluid is a liquid.

4. A device according to claim 3, wherein the said liquid is one which remains in liquid form after introduction into the balloon, the device further comprising an element for sealing the balloon after introduction of the liquid into the balloon.

5. A device according to claim 4, wherein the said liquid is selected from the group consisting of water and saline.

6. A device according to claim 4, wherein the said liquid is one which is capable of becoming a solid after introduction into the balloon.

7. A device according to claim 6, wherein the said liquid is capable of becoming a solid by application thereto of heat, the device further comprising a heating element for applying heat thereto.

8. A device according to claim 6, wherein the said liquid is capable of becoming a solid by application of light thereto, the device further comprising an element for applying light thereto.

9. A device according to claim 6, wherein the said liquid is a substance which becomes solid on addition of a hardener thereto, the device further comprising a source of hardener.

10. A device according to any preceding claim, comprising a locking system for holding the said tissue engaging member in engagement with the outwardly engaging surface of the second tissue layer.

11. A device according to claim 10, wherein the locking system comprises a resilient member for resiliently urging the said tissue engaging member into said engagement.

12. A device according to claim 10 or 11, wherein the locking system is arranged to provide locking at a plurality of different spacings between the balloon and the said tissue-engaging element.

13. A device according to any preceding claim, wherein the said tissue-engaging element is also an inflatable balloon.

14. A device according to any one of claims 1 to 12, wherein the said balloon and the said tissue-engaging member are both parts of a single balloon structure.

15. A device according to any one of claims 1 to 13, wherein the said inflation channel is defined within the said catheter.

16. A device according to claim 14, wherein the said inflation channel is separate from the catheter.

17. A method of joining together first and second tissue layers, which comprises passing through the layers the distal end portion of a catheter having a balloon mounted thereon, inflating the balloon and causing it to engage a surface of first tissue layer, and causing a tissue-engaging member to engage a surface of the second tissue layer, whereby the first and second tissue layers are urged into engagement with one another.
